(19) United States
(12) Patent Application Publication
(10) Pub. No.: US 2008/0254143 A1
(43) Pub. Date: Oct. 16, 2008

(54) COMPOSITION FOR IMPROVING IMMUNE SYSTEM HEALTH

(76) Inventors: MARVIN HEUER, MISSISSAUGA (CA); KEN CLEMENT, MISSISSAUGA (CA); CHAUDHURI SHAN, MISSISSAUGA (CA); MEGAN THOMAS, MISSISSAUGA (CA)

Correspondence Address: TORYS LLP, 79 WELLINGTON STREET WEST, SUITE 3000, BOX 270, TD CENTRE, TORONTO, ON, M5K 1N2, CA

(21) Appl. No.: 11/733,567

(22) Filed: Apr. 10, 2007

(51) Int. Cl. A61K 36/185; A61K 125/00; A61K 36/28; A61P 37/00; A61K 36/45; A61K 131/00

(52) U.S. Cl. 424/641; 424/732; 424/737; 424/777

(57) ABSTRACT

A composition and method for enhancing the immune system of an individual is provided. The present invention enhances the functioning of the immune system through increasing the production of certain cytokines, inhibiting viral adhesion and interfering in the viral replication process. The severity and duration of infections resulting from the common cold and associated viral infections are also reduced through the oral administration of the composition of the present invention. The composition of the present invention comprises at least an effective amount of an extract of wolfberry (Lycium barbarum), and an effective amount of an extract of elderberry. The present invention may additionally comprise effective amounts of one or more of an extract of blueberry, Echinacea root, and Vitamin C.

### FIELD OF THE INVENTION

The present invention relates to compositions for enhancing the immune system of an individual. More specifically, the present invention relates to a composition and a method comprising a synergistic combination of two fruit-based extracts for enhancing the immune system of an individual.

### BACKGROUND OF THE INVENTION

The immune system is a complex system of molecular and physical mechanisms within an organism responsible for protection of the body from infection. Through the identification of and subsequent neutralization followed by the removal of pathogens the immune system affords the body protection against infection. The Human immune system is based on two distinct forms of immunity, innate immunity and adaptive immunity. Pathogens are detected and distinguished from the organism's normal cells and tissue via complicated mechanisms involving the integration of both the innate and adaptive immune systems since pathogens are always evolving in order to avoid from detection. Such that the body can combat the dynamic nature of pathogens, vertebrates, such as humans have developed immune systems consisting of many types of proteins, cells, organs, and tissues that interact in an elaborate network (Beck G, Habicht G S. Immunity and the Invertebrates. Scientific American November 1996:60-66).

The innate immune response is the non-specific response against non-viral infections mounted by the immune system and is characterized by the infiltration of cells such as leukocytes such as natural killer cells, masT-cells, eosinophils and basophils as well as phagocytes such as neutrophils, macrophages and dendritic cells in response to chemotatic signaling at the site of injury or infection. Molecules secreted by these aforementioned cells such as histamine and various cytokines; and the complement system of circulating proteins assist several aspects of the immune response, however they contribute to inflammation through the cellular signaling for infiltration of immune cells to the cell of injury or infection (Kawai T, Akira S. Innate immune recognition of viral infection. Nat. Immunol. 2006;7(2): 131-7). There are several common clinical symptoms associated with inflammation and the innate immune system including pain, localized redness, swelling and heat at the site of inflammation.

The adaptive immune system, on the other hand is composed of highly specialized, systemic cells and processes that eliminate pathogenic challenges. It is this immune response that makes the human immune system able to recognize and remember specific pathogens, such as a viruses. This generated immunity makes the immune system capable of mounting stronger attacks each time the pathogen is encountered. Development of immunological memory is based upon the expression of signature antigens on individual lymphocytes such as B and T-cells (Pancer Z, Cooper M. The evolution of adaptive immunity. Annu Rev Immunol 2006;24:497-518). Therefore, by increased proliferation of these cell types can result in a more immediate and specific immune response.

Molecules, secreted by immune cells, such as histamine and various cytokines, and the complement system of circulating proteins assist several aspects of the immune response. Specifically, cytokines such as interleukins have anti-viral effects, such as shutting down protein synthesis in the hosT-cell. It is possible to add compounds which can directly interfer with viral synthesis, thereby limiting the propagation of virus in nearby cells and thus aiding the immune system.

### SUMMARY OF THE INVENTION

The present invention is directed towards an orally administered immune-system enhancing composition comprising an effective amount of an extract of wolfberry (Lycium barbarum), and an effective amount of an extract of elderberry. In additional aspects of the present invention, one or more of an extract of blueberry, Echinacea root, vitamin C, and zinc sulphate or zinc acetate are added to the composition to provide further synergistic or additional benefits. Both a composition and a method are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards an orally administrable immune-system enhancing composition, comprising effective amounts of two fruit-based extracts. The present invention comprises at least an extract of wolfberry (Lycium barbarum), and an extract of elderberry. According to various embodiments, the present invention may further comprise an extract of blueberry, Echinacea root, vitamin C, and zinc sulphate or zinc acetate. Wolfberry

Wolfberries, the common name for Lycium barbarum, also called Goji berries are a nutritionally rich fruit originally grown in Europe and now cultivated in China. They contain all 8 essential amino acids, as well as 21 trace minerals and a variety of vitamins. In traditional Chinese medicine they have been used in for nearly 2,000 years. It is understood that the active polysaccharide extracts provide benefits such as enhancing the immune system function, improving eyesight, protecting the liver, boosting sperm production, and improving circulation in an individual.

The use of Wolfberry polysaccharides in the treatment of age-related oxidative stress in mice has been shown to improve the total antioxidant capacity as well as improved immune function (Li X M, Ma Y L, Liu X J. Effect of the Lycium barbarum polysaccharides on age-related oxidative stress in aged mice. J Ethnopharmacol. Dec. 28, 2006; [Epub ahead of print (Abstract)). Furthermore, the same study also illustrated that the antioxidant activity of the wolfberries could be increased with co-treatment of vitamin C.

Administration of Lycium barbarum polysaccharides was also shown to increase the expression of interleukin-2 (IL-2) and tumor necrosis factor-alpha (TNF-α) at both mRNA and protein levels in a dose-dependent manner (Gan L, Zhang S H, Liu Q, Xu H B. A polysaccharide-protein complex from Lycium barbarum upregulates cytokine expression in human peripheral blood mononuclear cells. Eur J Pharmacol. Jun. 27, 2003;471(3):217-22(Abstract)). These results suggest that Lycium barbarum polysaccharides may induce immune responses and possess potential therapeutic efficacy in cancer.

Interleukin-2 (IL-2) is a protein belonging to the immune system that is instrumental in the body's natural response to microbial infection. Furthermore, it aids in the discrimination between foreign (nonself) and self matter in the body. IL-2 confers its effects by binding to IL-2 receptors, which are cell-surface receptors expressed by lymphocytes, the cells that are responsible for immunity through their production of antibodies. Additionally, IL-2 is responsible for stimulating the growth, inducing differentiation and survival of antigen-selected cytotoxic T-cells via the activation of the expression of specific genes. As such, IL-2 is necessary for the development of T-cell immunologic memory, which depends upon the expansion of the number and function of antigen-selected T-cell clones.

It is herein understood by the inventors that the incorporation of Lycium barbarum polysaccharides in an immunity-enhancing composition will effectively increase expression of IL-2 and TNF-α. This increased expression will result in an enhanced immune response to infection.

In an embodiment of the present invention, which is set forth in greater detail in the examples below, the immune system enhancing supplement includes an extract of wolfberry. A serving of the immune system enhancing supplement includes from about 0.00001 g to about 1.0000 g of an extract of wolfberry. The preferred dosage of a serving of the immune system enhancing supplement comprises about 0.001 g of an extract of wolfberry.

### Elderberry

Elderberries are the fruit of the black elder tree (Sambucus nigra) which is native to in Europe. Rich in bioflavanoids and anthocyanins, they have been used for centuries in folk medicine. Recently Elderberry extracts have been shown to influence cell function and provide immune system support.

Elderberry syrup given to patients as a 15 ml serving, four times daily for a period of 5 days (Brown D J, Standardized elderberry syrup shortens the severity and duration of influenza in adults. HerbalGram. 2004;63:16-7), showed attenuation in both the severity and the duration of influenza infections. The reduction in the duration of influenza infections as shown in these studies has been purported to be a result of an increase in the production of cytokines, mainly TNF-α.

Additionally, extracts of elderberry have been shown to inhibit the replication of common influenza A and B strains as well as and through the prevention of viral adhesion to cell receptors (Zakay-Rones Z, Varsano N, Zlotnik M, Manor O, Regev L, Sclesinger M, Mumcuoglu M. Inhibition of several strains of influenza virus in vitro and reduction of symptoms by an elderberry extract (Sambucus nigra L.) during an outbreak of influenza B Panama. J Altern Complement Med. 1995 Winter;1(4):361-9). This prevention of viral adhesion to cell receptors makes elderberry viable choice for a herbal treatment of rhinovirus infections, the common cold.

It is herein understood by the inventors that the incorporation of an extract of elderberry in an immune system enhancing supplement will result in an increase in the production of cytokines and inhibition of viral adhesion to cell receptors as well as viral replication. The combination of these two effects will result in a shorter and less severe period of a rhinovirus infection in an individual.

In an embodiment of the present invention, which is set forth in greater detail in the examples below, the immune system enhancing supplement includes an extract of elderberry. A serving of the immune system enhancing supplement includes from about 0.0001 g to about 1.000 g of an extract of elderberry. The preferred dosage of a serving of the immune system enhancing supplement comprises about 0.0010 g of an extract of elderberry.

### Blueberry Extract

Blueberries are a group of flowering plants in the genus Vaccinium. The species are native to North America and eastern Asia and contain a high amount of anthocyanins, potent antioxidants.

Anthocyanins are water-soluble flavonoid pigments that appear red to blue in color depending upon the pH. Flavonoids have been reported to demonstrate their benefits in lowering oxidative stress and also have beneficial effects on cardiovascular and chronic inflammatory diseases.

Anthocyanins and anthocyanin-rich extracts were found to induce TNF-α production and act as modulators of the immune response in activated macrophages (Wang J, Mazza G. Effects of anthocyanins and other phenolic compounds on the production of tumor necrosis factor alpha in LPS/IFN-gamma-activated RAW 264.7 macrophages. J Agric Food Chem. Jul. 17, 2002;50(15):4183-9 (Abstract)). TNF-α is a cytokine that is involved in systematic inflammation and plays a major role in the regulation of immune cells. TNF-α potently attracts neutrophils and assists their adhesion to the endothelial cells to induce migration. The action of TNF-α on macrophages stimulates phagocytosis and the production of IL-1 oxidants and the inflammatory lipid, prostaglandin.

It is herein understood by the inventors that the incorporation of a known enhancer of TNF-α in an immune system enhancing composition will yield a faster immune response as compared to in the absence of said composition, resulting decreased likelihood of an individual presenting symptoms of an infection or a shorter period of infection.

In an embodiment of the present invention, which is set forth in greater detail in the examples below, the immune system enhancing supplement includes an extract of blueberry. A serving of the immune system enhancing supplement includes from about 0.0001 g to about 1.0000 g of an extract of blueberry. The preferred dosage of a serving of the immune system enhancing supplement comprises about 0.0010 g of an extract of blueberry.

### Echinacea Extract

Echinacea refers to a genus consisting of nine species of flowering plants, which are all native to eastern North America. All species of Echinacea possess compound phenols, which are often purported to be responsible for Echinacea's health benefits. Most notable of theses health benefits is the ability to boost the body's immune system and ward off infections, particularly to the common cold.

The effect of treatment with Echinacea tea on cold symptoms was studied in a randomized, double-blind placebo-controlled study (Lindenmuth G F, Lindenmuth E B. The efficacy of echinacea compound herbal tea preparation on the severity and duration of upper respiratory and flu symptoms: a randomized, double-blind placebo-controlled study. J Altern Complement Med. August 2000;6(4):327-34 (Abstract)). This study illustrated that treatment with Echinacea tea was effective for relieving cold symptoms in a shorter period of time then the group treated with the placebo.

Additionally, a meta-analysis has suggested that standardized extracts of Echinacea were effective in the prevention of symptoms of the common cold after clinical inoculation, in comparison to the placebo (Schoop R, Klein P, Suter A, Johnston S L. Echinacea in the prevention of induced rhinovirus colds: a meta-analysis. Clin Ther. February 2006;28(2):174-83 (Abstract)). It is herein understood by the inventors that the incorporation of Echinacea root in an immune system enhancing supplement, will help to prevent the prevalence of rhinovirus infection symptoms in an individual. Furthermore, it is understood that incorporation of Echinacea root, in an immune system enhancing supplement, will help to reduce the duration and severity of rhinovirus infection in an individual.

In an embodiment of the present invention, which is set forth in greater detail in the examples below, the immune system enhancing supplement comprises Echinacea root. A serving of the immune system enhancing supplement includes from about 0.001 g to about 1.000 g of Echinacea root. The preferred dosage of a serving of the immune system enhancing supplement comprises about 0.0250 g of Echinacea root.

### Vitamin C

Vitamin C, or L-ascorbic acid, is an essential nutrient required in small amounts in order to allow a range of essential metabolic reactions in animals and plants. Since the body does not make or store vitamin C, humans are required to ingest it in their daily diets. Ascorbic acid is a strong antioxidant and protects the body from oxidative stress, as well as acting as a coenzyme in necessary enzymatic reactions. As a strong antioxidant and anti-inflammatory molecule, ascorbic acid has been shown to protect against the common cold.

Vitamin C concentrations in plasma and leukocytes rapidly decline during infections and stress, therefore supplementation during periods of infection is desired in order to maintain essential metabolic reactions. Supplementation of vitamin C is known to improve antimicrobial and natural killer cell activities, as well as increase lymphocyte proliferation. Studies of the role of vitamin C in immune response situations have concluded that ascorbic acid helps to protect cells from reactive oxygen species that are produced during immune responses (Wintergerst E S, Maggini S, Hornig D H. Immune-enhancing role of vitamin C and zinc and effect on clinical conditions. Ann Nutr Metab. 2006;50(2):85-94. Epub Dec. 21, 2005. Review).

In a meta-analysis it was concluded that daily supplementation of ascorbic acid, in amounts greater then 100 mg, lowers the incidence, severity and integrated morbidity of the common cold (Pauling L. The significance of the evidence about ascorbic acid and the common cold. Proc Nat Acad Sci USA November 1971;68(11):2678-81). It is herein understood by the inventors that the incorporation of ascorbic acid in an immune system enhancing supplement will effectively reduce incidence, severity and duration of cold related infections as well as help to maintain vitamin C levels in an individual during infections.

In an embodiment of the present invention, which is set forth in greater detail in the examples below, the immune system enhancing supplement includes vitamin C. A serving of the immune system enhancing supplement includes from about 0.100 g to about 2.000 g of vitamin C. The preferred dosage of a serving of the immune system enhancing supplement comprises about 1.100 g of vitamin C.

### Zinc (Acetate and Sulphate)

Zinc is an essential element, necessary for sustaining all life. It is estimated that 3000 of the hundreds of thousands of proteins in the human body contain zinc prosthetic groups. Zinc is required for a variety of physiological reactions including the production, growth and functioning of immune system cells.

A review of laboratory and clinical evidence supported the value of zinc for reducing the duration and severity of symptoms of a common cold when administered within 24 hrs of the onset of symptoms (Hulisz D. Efficacy of zinc against common cold viruses: an overview. J Am Pharm Assoc (Wash D.C.). September-October 2004;44(5):594-603 (Abstract)). In terms of Zinc's mechanism of action it has been shown to prevent the formation of viral capsid proteins and thus inhibits in vitro replication of the rhinovirus (Korant B D, Butterworth B E. Inhibition by zinc of rhinovirus protein cleavage: interaction of zinc with capsid polypeptides. J Virol. April 1976;18(1):298-306). When sufficient amounts of zinc bind directly to a site in the rhinovirus capsid protein and alter its conformation such that it cannot undergo the requisite cleavage reactions an interruption in the replication of the viruses occurs. This helps to reduce the spread of the infection to nearby cells as well inhibit the replication of the virus.

Both Zinc acetate and Zinc sulphate have been shown to reduce the duration and overall severity of cold symptoms in volunteers recruited within 24 hours of developing cold symptoms (Prasad A S, Fitzgerald J T, Bao B, Beck F W, Chandrasekar P H. Duration of symptoms and plasma cytokine levels in patients with the common cold treated with zinc acetate. A randomized, double-blind, placebo-controlled trial. Ann Intern Med. Aug. 15, 2000;133(4):245-52; Kurugol Z, Akilli M, Bayram N, Koturoglu G. The prophylactic and therapeutic effectiveness of zinc sulphate on common cold in children. Acta Paediatr. October 2006;95(10):1175-81 (Abstract)).

It is herein understood by the inventors that the incorporation of Zinc acetate, Zinc sulphate, or mixture thereof in an immune system enhancing supplement will inhibit the replication and spread of viruses within the body, resulting in reduction of the duration and severity of viral infections.

In an embodiment of the present invention, which is set forth in greater detail in the examples below, the immune system enhancing supplement includes Zinc acetate, Zinc sulphate, or mixture thereof. A serving of the immune system enhancing supplement includes from about 0.001 g to about 0.100 g of Zinc acetate, Zinc sulphate, or mixture thereof. The preferred dosage of a serving of the immune system enhancing supplement comprises about 0.015 g of Zinc acetate, Zinc sulphate, or mixture thereof.

In an embodiment of the present invention, which is set forth in greater detail Example 1, the immune-system enhancing composition comprises an extract of wolfberry (Lycium barbarum), an extract of elderberry, an extract of blueberry, Echinacea root, vitamin C, and zinc sulphate or zinc acetate. The composition is provided in any acceptable and suitable oral dosage form as known in the art to attenuate the intensity and duration of an inflammatory reaction as a result of infection or injury, reduce the immune-mediated cell damage associated with infections and inhibit the viral replication process. The composition may also be provided in a prolonged or extended release format.

While, not wishing to be bound by theory, present invention is comprised of components that have been shown to increase the expression of IL-2 and TNF-α at both mRNA and protein levels. It is herein understood by the inventors that increased TNF-α and IL-2 expression will effectively enhance immune response to infection.

Furthermore, the present invention comprises components that have been shown to increase in the production of cytokines, mainly TNF-α, as well as prevent viral adhesion to cell receptors (Zakay-Rones Z, Varsano N, Zlotnik M, Manor O, Regev L, Sclesinger M, Mumcuoglu M. Inhibition of several strains of influenza virus in vitro and reduction of symptoms by an elderberry extract (Sambucus nigra L.) during an outbreak of influenza B Panama. J Altern Complement Med. 1995 Winter;1(4):361-9). It is herein understood by the inventors that an increase in the production of cytokines and inhibition of viral adhesion to cell receptors as well as inference in the viral replication process will reduce the possibility of infection, reduce the severity of infections and shorten the periods of infection.

Further to the aforementioned function, the present invention comprises components that have been shown to bind directly to a site in viral capsid protein and alter its conformation so that it cannot undergo the requisite cleavage reactions. It is herein understood by the inventors that inhibition of the formation of viral capsid protein will limit viral replication.

The present invention is understood by the inventors to increase the effectiveness of the immune system of an individual in response to infection by pathogens by substantially simultaneously affecting several mechanisms of the immune system as herein disclosed.

According to various embodiments of the present invention, the immune-system enhancing supplement may be consumed in any form. For instance, the dosage form of the immune-system enhancing supplement may be provided as, e.g., a powder beverage mix, a liquid beverage, a ready-to-eat bar or drink product, a capsule, a liquid capsule, a tablet, a caplet, an effervescent tablet, or as a dietary gel. The preferred dosage form of the present invention is as an effervescent tablet.

Additionally, by way of ingestion of the composition of the present invention, a method for enhancing the effectiveness of the immune system in an individual is provided. The method of the present invention comprises at least the step of administering to an individual an effective acceptable amount of the composition of the present invention.

Furthermore, the dosage form of the immune-system enhancing supplement is provided in accordance with customary processing techniques for herbal and nutritional supplements in any of the forms mentioned above. Additionally, the immune-system enhancing supplement set forth in the example embodiment disclosed herein may contain any appropriate number and types of excipients as is well known in the art.

Although the following example illustrates the practice of the present invention in two of its illustrative compositional embodiments, the examples should not be construed as limiting the scope of the invention. Other embodiments will be apparent to one of skill in the art from consideration of the specifications and example.

### EXAMPLES

### Example 1

An immune-system enhancing supplement comprising the following ingredients per serving is prepared for consumption as an effervescent tablet:

about 0.001 g of an extract of wolfberry (Lycium barbarum), and about 0.001 g of an extract of elderberry

### Example 2

An immune-system enhancing supplement comprising the following ingredients per serving is prepared for consumption as an effervescent tablet:

about 0.001 g of an extract of wolfberry (Lycium barbarum), about 0.001 g of an extract of elderberry, about 0.050 g Echinacea root and about 1.100 of vitamin C.

### Example 3

An immune-system enhancing supplement comprising the following ingredients per serving is prepared for consumption as an effervescent tablet:

about 0.001 g of an extract of wolfberry (Lycium barbarum), about 0.001 g of an extract of elderberry , about 0.025 g Echinacea root, about 0.015 g of Zinc and about 1.100 of vitamin C.

### Extensions and Alternatives

In the foregoing specification, the invention has been described with a specific embodiment thereof, however, it will be evident that various modifications and changes may be made thereto without departing from the broader spirit and scope of the invention.

1. A composition comprising an effective amount of an extract of wolfberry (Lycium barbarum), and an extract of elderberry for enhancing the immune system of a mammal.

2. The composition of claim 1 wherein the extract of wolfberry (Lycium barbarum) and an extract of elderberry act synergistically through differing molecular mechanisms to enhance the immune system.

3. The composition of claim 2 wherein the extract of wolfberry (Lycium barbarum) increases the expression of IL-2 and TNF-α.

4. The composition of claim 2 wherein the extract of elderberry increases in the production of cytokines and prevent viral adhesion to cell receptors.

5. The composition of claim 1, further comprising an effective amount of an extract of blueberry.

6. The composition of claim 5 wherein the extract of blueberry induces TNF-α production and acts as a modulator of activated macrophages.

7. The composition of claim 1, further comprising an effective amount of Echinacea root.

8. The composition of claim 7 wherein the Echinacea extract reduces occurrence, severity and duration of an infection in a mammal.

9. The composition of claim 1, further comprising an effective amount of a source of vitamin C.

10. The composition of claim 9 wherein the vitamin C reduces occurrence, severity and duration of infection.

11. The composition of claim 9 wherein the vitamin C increases the antioxidant effect of wolfberry extract.

12. The composition of claim 1, further comprising an effective amount of zinc.

13. The composition of claim 12 wherein the zinc interferes with viral capsid formation thereby limiting viral replication.

14. The composition of claim 12 wherein said zinc is selected from the group consisting of zinc acetate and zinc sulphate.

15. The composition of claim 1, further comprising and effective amount of zinc acetate.

16. The composition of claim 15 wherein the zinc acetate interferes with viral capsid formation thereby limiting viral replication.

17. A method for enhancing the immune system in a mammal, the method comprising the step of administering to said mammal a composition comprising an effective amount of an extract of wolfberry (Lycium barbarum) and an effective amount of an extract of elderberry.

18. The composition of claim 17 wherein the extract of wolfberry (Lycium barbarum) and an extract of elderberry act synergistically through differing molecular mechanisms to enhance the immune system.

19. The composition of claim 18 wherein the extract of wolfberry (Lycium barbarum) increases the expression of IL-2 and TNF-α.

20. The composition of claim 18 wherein the extract of elderberry increases in the production of cytokines and prevent viral adhesion to cell receptors.

21. The composition of claim 17, further comprising an effective amount of an extract of blueberry.

22. The composition of claim 21 wherein the extract of blueberry induces TNF-α production and acts as a modulator of activated macrophages.

23. The composition of claim 17, further comprising an effective amount of Echinacea root.

24. The composition of claim 23 wherein the Echinacea extract reduces the occurrence, severity and duration of an infection in a mammal.

25. The composition of claim 17, further comprising an effective amount of a source of vitamin C.

26. The composition of claim 25 wherein the vitamin C increases reduce occurrence, severity and duration of infection.

27. The composition of claim 25 wherein the vitamin C increases the antioxidant effect of the wolfberry extract.

28. The composition of claim 17, further comprising an effective amount of zinc.

29. The composition of claim 28 wherein the zinc sulphate interferes with viral capsid formation thereby limiting viral replication.

30. The composition of claim 28 wherein the zinc is selected from the group consisting of zinc acetate and zinc sulphate.

31. The composition of claim 1 wherein the composition is provided in an effervescent tablet.

32. The method of claim 17 wherein the composition is provided in an effervescent tablet.

33. The composition of claim 1 wherein the composition is provided in an orally acceptable dosage format selected from the group consisting of a tablet, a caplet, a dietary gel and capsule.

34. The method of claim 17 wherein the composition is provided in an orally acceptable dosage format selected from the group consisting of a tablet, a caplet, a dietary gel and capsule.
